(19) United States
(12) Patent Application Publication
(10) Pub. No.: US 2022/0226094 A1
(43) Pub. Date: Jul. 21, 2022

(54) METHOD USING CUSTOM PATIENT SPECIFIC IMPLANTS TO DECREASE THE NATURAL RATE OF AGING SITE SPECIFIC REGIONS OF THE FACIAL SKELETON

(71) Applicants: Gregory C. CHOTKOWSKI, Nantucket, MA (US); Gwendolyn S. REEVE, Fairfield, CT (US)

(72) Inventors: Gregory C. CHOTKOWSKI, Nantucket, MA (US); Gwendolyn S. REEVE, Fairfield, CT (US)

(21) Appl. No.: 17/577,494

(22) Filed: Jan. 18, 2022

Related U.S. Application Data
(60) Provisional application No. 63/138,955, filed on Jan. 19, 2021.

(51) Int. Cl. A61F 2/00; G06F 30/10

(52) U.S. Cl. CPC A61F 2/0059; G06F 30/10

(57) ABSTRACT

Custom facial implants are used in a method to decrease and arrest the natural rate of aging in site specific areas of the facial skeleton in an individual by utilizing these custom facial implants in site specific areas, including the steps of: using CADCAM and 3D printing to create the custom patient facial implants; placing the custom implants on the site-specific areas of the facial skeleton through a transoral approach such that there are no external incision or scars on the patients face; such that the custom implants are surgically fixated directly to the bone and promotes integration of the implant to the underlying bone to prevent future changes to the facial skeleton in the specific facial areas to decrease or arrest the natural aging process such that the overlying soft tissue of the facial region is supported and resuspended by the custom facial implant

This application claims the benefit of U.S. provisional application Ser. No. 63/138,955 filed Jan. 19, 2021 which is incorporated herein in its entirety by reference.

### FIELD OF THE INVENTION

A prediction method that decreases or arrests the appearance of the natural aging process of site-specific areas of the facial skeleton to restore a youthful appearance by adding volume and resuspending the overlying facial tissue with a manufactured custom facial implant.

### BACKGROUND OF THE INVENTION

Individuals want to maintain their youthful appearance, creating a need for implantable fillers and newer surgical techniques to address the natural aging process of the face. This aging process includes increased laxity of the skin, loss and redistribution of facial fat volume and sagging of these tissues that results in folds and wrinkles of the skin. In addition, researchers have identified that certain site-specific areas of the facial skeleton also undergo resorptive changes, which result in apparent soft tissue volume loss. A published research paper by Bryan Mendelson⋅Chin-Ho Wong Changes in the Facial Skeleton With Aging: Implications and Clinical Applications in Facial Rejuvenation Aesth Plast Surg (2012) 36:753-760 DOI 10.1007/s00266-012-9904-3 details these site-specific areas of the facial skeleton that undergo the most resorptive bony changes in aging and must be addressed to obtain a natural-appearing facial rejuvenation.

Most individuals undergoing a cosmetic procedure would like to know what they will look like after the procedure before having the actual procedure. Currently, there are software applications on the market that are available to surgeons performing cosmetic procedures. The surgeon will utilize the software to give the patient an understanding of what they could look like after the procedure by creating a prediction image. These are not true prediction software applications; the applications do not provide an accurate surgical result because they cannot rely on the surgeon's skill and the surgical techniques used to perform these procedures. Furthermore, the software application is not directly utilized during the procedure itself. In most cases, the final procedural results are different from the prediction image. But these prediction software applications serve as an educational tool in allowing an individual to understand the limitations of a particular surgical procedure and of the surgeon.

Illustrative of these known methods include U.S. Pat. No. 9,250,620 to Koltlus, which discloses a 3D design and fabrication system for corrective implants.

U.S. Pat. Nos. 7,747,305, 9,208,558, 9,292,920, 9,672,302, 9,672,617 and 10,068,671 all to Dean et al. are related to computer-aided design method for producing implants using 3D scans of patients and generating implant design data generated on the computer. While it appears that these patents, in general, aim to correct cranial and skeletal defects, they do not suggest delaying or stopping the appearance of the aging process or their uses in facial cosmetic surgery.

U.S. Pat. No. 7,953,260 to Weinzweig et al. discloses a method of predicting the movement of the face's soft tissue in response to the movement of the underlying bone and using data to predict for a second subject movement of the soft tissue of the face over time in response to movement of the underlying bone.

The facial skeleton is the framework that supports the overlying soft tissue. By restoring the bone loss in site specific areas of the facial skeleton that undergo age-related resorption, the invention provides a method to resuspend and add volume to the overlying deficient and sagging soft tissue with a custom facial implant. The invention facial implant is custom designed for each individual and addresses reversing the aging process in several critical aesthetic zones of the face.

It is viable that placing these custom implants over the area of the facial skeleton that has undergone bone resorption and fixating them to the underlying bone will decrease or arrest further resorption of the facial skeleton in these identified areas.

The prediction methodology ensures that the implant is designed and manufactured to restore the lost bone volume that results from aging and allows an opportunity to enhance facial features for individuals who have a preexisting bony deficiency or facial asymmetry. This custom implant can be designed to replace both the bone and soft tissue volume and at the same time resuspend the overlying soft tissue.

The goal of the invention of the custom facial implants and prediction methodology application is to use the information from both the preoperative facial CT scan and overlay the aesthetically enhanced 3D image and, through the soft tissue prediction algorithm and subtraction of the two images, design the custom facial implant. A catalog of implants presented on a skeletal model will contain the outcome of the area-specific custom facial implants. The model will serve as the ideal aesthetic proportions for both males and females.

The prediction methodology will, through an algorithm, automatically provide an enhancement for the area[s] of interest[s] for each individual. For example, if a woman would like to enhance her cheekbones, she would select “cheekbone” from the implant catalog, and the software program will provide her imagery of a desirable cheekbone with overlying soft tissue. This cheekbone would be proportional to this individual based on an algorithm created to reference the imagery of [models, celebrities] aesthetically pleasing individuals. The algorithm will also consider the patient's age, weight and soft tissue thickness in these critical aesthetic zones of the face.

As part of the invention, there is a separate software methodology that will map out, from a 3D video, the proportions of these aesthetically pleasing model individuals. Known anatomical landmarks—i.e., the location of the pupil, the lateral canthus, bridge of nose, tip of nose angle of mandible etc.—will be the foundation for mapping an aesthetically pleasing cheekbone. This algorithm will then be used on the specific individual and suggest a proportional and aesthetically pleasing cheekbone. The individual will then have the opportunity to refine the suggested cheekbone themselves with easy-to-use software drawing and mesh tools. Patient participation in the treatment planning typically increases overall patient satisfaction.

The invention accomplishes these objectives by utilizing a methodology algorithm wherein each individual who receives a custom facial implant will undergo a CT scan and obtain a 3D facial photo. The algorithm considers many variables including but not limited to age, skin type, soft tissue thickness and laxity, which will dictate the shape and volume of the custom facial implant. The algorithm will be applied to the 3D rendering of the facial skeleton and the 3D facial photo, allowing the prospective patient to view a 3D soft tissue facial rendering of what they may look like following placement of the custom facial implant.

Alternatively, the individual can dictate their desired esthetic outcome by manipulating the software, and the programmed subtractive algorithm will determine the desired implant's volume and shape. The algorithm will map out the implant's volume and anatomic positions of the implant and calculate the difference of the soft tissue projection. These values will refine the soft tissue prediction software utilized in the custom facial implant design. In turn, the creation and placement of each future implant will provide valuable information that will make the prediction software application and design of the custom facial implant more accurate for the next individual utilizing this invention.

A general objective of the invention method is that it may decrease or arrest the aging process in site specific areas of the facial skeleton.

Another objective of the invention is to provide a method that surpasses the current practice of injecting fillers into soft tissues to restore volume; This invention is more advantageous since it does not reabsorb and provides a more permanent solution. Preferred applications are to the face and jaws: cheekbones, piriform region, mandible (angle and chin), and jawbone facial areas. An advantage of the implant compared to facial fillers is a surgeon can remove the implant if the patient is unsatisfied with the outcome from the placement of the custom facial implant. Facial fillers are not easy to remove once injected.

Yet another method of the invention is to provide a prediction method or “morphing tool” that allows the individual to see what they would look like after placement of the custom facial implant by using a three-dimensional facial image or augmented reality.

Still another objective of the invention is to provide a custom facial implant that may include a hook or barb on the soft tissue surface side of the implant. These anchorage points will be designed and incorporated onto the soft tissue surface of the implant to support and resuspend the soft tissue overlying the implant. Hooks, barbs or projections may be designed and incorporated on the bony surface of the implant to assist in the retention and stabilization of the custom facial implant when placed on the facial skeleton.

### SUMMARY OF THE INVENTION

In the present invention, these purposes and others provide a method of decreasing and arresting the aging process of site-specific areas of the facial skeleton by utilizing custom facial implants. The custom facial implants are created using CADCAM and 3D printing.

These custom facial implants are placed on the site-specific areas of the facial skeleton through a transoral approach, leaving no external incisions or scars on the patient's face. This procedure can take place in an office, hospital or ambulatory surgery center under IV sedation or general anesthesia. The recovery time should be only a week with immediate results. This procedure does not require external incisions and will not leave scars on the patient's face. The custom facial implants are fixated directly to the underlying bone and may prevent future changes to the facial skeleton in said areas to decrease and arrest the natural aging process and restore lost facial volume.

The custom facial implants will not resorb like bone, and the bone underneath the implants will be maintained by bony integration of the implant thereby decreasing and arresting the aging process or the resorption of bone in site specific areas of the facial skeleton. The implants add volume and resuspend the soft tissue above it, adding volume to the areas where facial fat has been lost and redistributed, and where the facial skin has sagged. Many individuals are born with bony deficiency in the critical esthetic zones of the facial skeleton; they have less of a foundation to support the overlying soft tissue. Bony deficiencies lead to the appearance of early and more accelerated facial aging. Restoring the foundation through the invention methodology and prediction software application provides a lift to the patient's face giving them a more youthful appearance. Other objects, features, and advantages of the present invention will be apparent when considering the detailed description of the invention's preferred embodiments with reference to the drawings, which are illustrative, not limiting.

### BRIEF DESCRIPTION OF THE DRAWINGS

FIG. 1 is a schematic illustration of the process steps of the invention methodology.

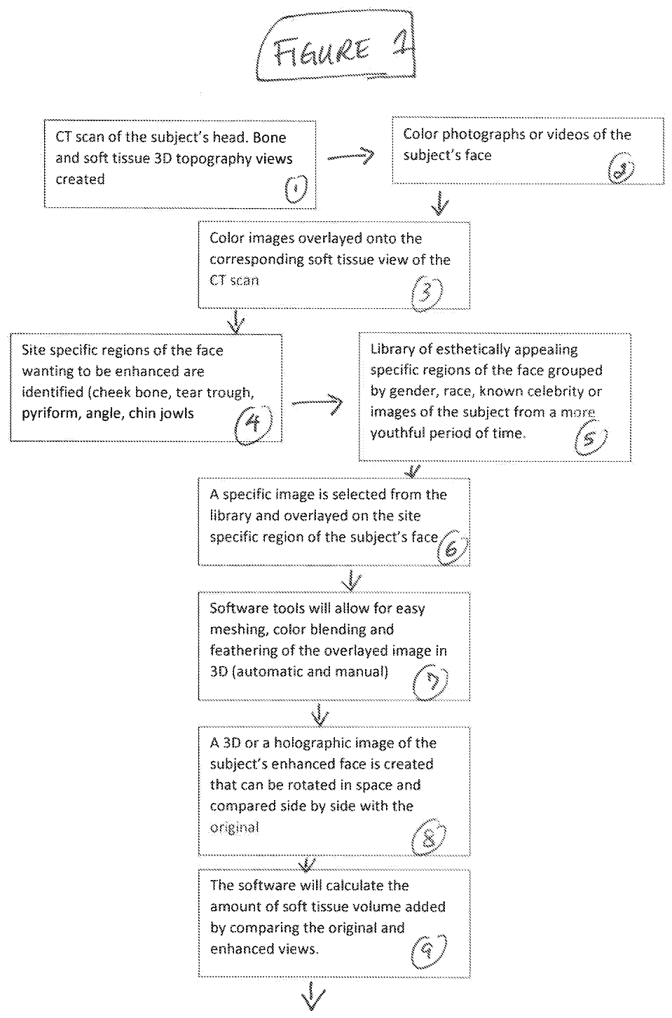

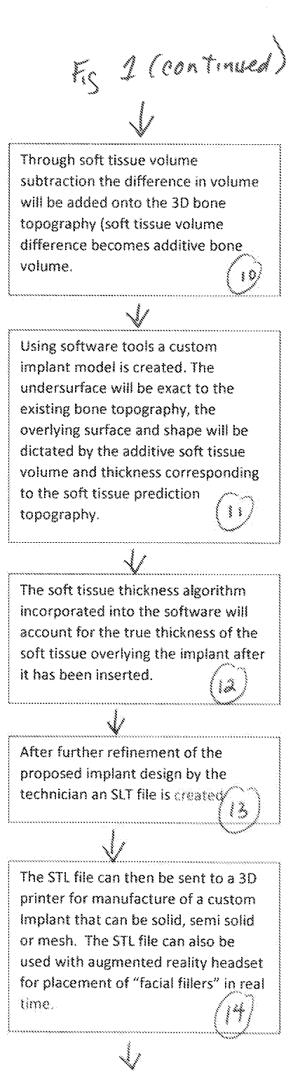

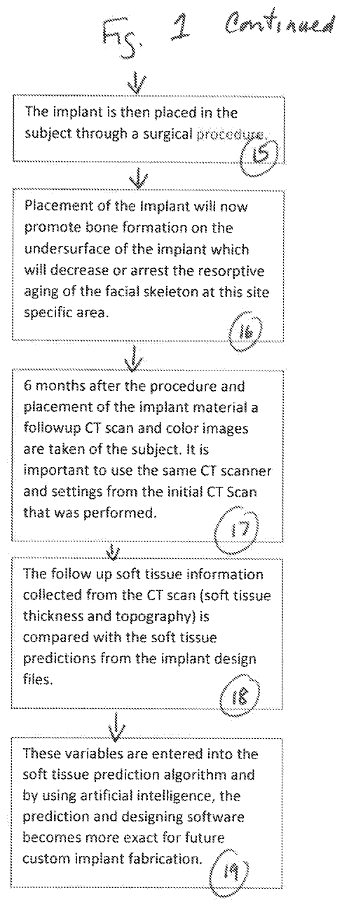

### DETAILED DESCRIPTION OF THE INVENTION

In accordance with the drawings, the present invention provides a method to decrease or arrest the appearance of the aging process in site specific areas of the facial skeleton and to restore the overall youthful appearance by adding volume and resuspending the overlying facial soft tissue using a printed or CAD CAM manufactured custom facial implant. A software algorithm is created by the process by which each patient that receives a custom facial implant will be scanned and the post implant soft tissue enhancement will be compared to the original soft tissue. The software algorithm will map out the volume of the implant and the anatomic position of the implant to calculate the projected difference of the overlying soft tissue. These values will refine the soft tissue prediction software utilized in the custom facial implant design. In turn, the creation and placement of each future implant will provide valuable information that will make the prediction software and design of the custom facial implant more accurate going forward. An individual interested in this particular method of facial rejuvenation will use photographic images to provide a visual enhancement through the use of the prediction software. The individual will be able to identify certain predefined regions of the face and make enhancements to their appearance using simple software tools. The critical esthetic areas of the face are the angle of the mandible, the chin and Jowl areas, the nasolabial folds, tear trough, cheekbone, and lateral orbital rims. The patient will have a CT scan of their face. The CT will provide a high-resolution 3D image of the bony topography and the overlying soft tissue. Using another feature of the prediction software will wrap the color imported image of the individual on top of the 3D CT generated soft tissue model. The individual will select a specific predefined region of the face for enhancement and visually see the results in real-time. Through subtraction of the desired soft tissue prediction and the original soft tissue topography, the software application will design a custom facial implant that will lie exactly and passively on the contour of the underlying bone. A technician will review the custom facial implant design for further refinements, such as feathering of sharp edges, avoiding critical anatomical structures, extending the implant to provide additional surface area for ideal bony seating and placement. An STL file will be created for the proposed implant(s). The implants will then be fabricated, packaged, and sent to the surgeon. The surgeon will be responsible for sterilizing the implant before inserting. Sterilizing requires an in-office autoclave machine, set to the implant manufacturer's specification. In the future, when the demand for custom facial implants increases and the cost of the 3D printers decreases, the surgeon will have the option to design and manufacture the implants in their office, saving both time and production costs.

The preferred implant material for custom facial implants is a non re absorbable material. The material used in the invention should be FDA approved and safe for human implantation. Material according to the invention includes PEKK (Polyetherketoneketone). This material has many beneficial characteristics; however, materials currently available or in development or ones that may be identified in the future will also work with this method. OPM (Oxford Performance Materials in Connecticut) currently manufactures custom facial implants. They have FDA approval for 3D printing of non-load bearing facial implants using PEKK (Osteofab). The main characteristics of PEKK, compared to the commonly used implantable materials such as PEEK and titanium, is that PEKK has research supported antibacterial properties and promotes long-term bony integration on its uniquely engineered surface. The implants can easily be adjusted at the time of surgery if necessary.

The procedure will take place in an outpatient setting, in Office, Ambulatory Surgical Center or Hospital. The procedure will be performed under General Anesthesia or IV sedation. The surgery is performed trans-orally (through an incision in the mouth); a subperiosteal dissection will be performed, creating a pocket for the implant along with elevating the overlying soft tissue. The implant will be placed in this pocket and lie passively on the bony surface. The surgeon will place one or two fixation screws through predrilled holes in the implant (the holes will be designed to avoid critical anatomic structures and allow the screws to be placed in the thickest bone of the facial skeleton under the implant). Once the implants are securely in place, the site is irrigated with copious saline irrigation or antibiotic irrigation. The external soft tissue is then checked for any puckering (caused by tethered down or trapped deep tissue. This is then freed with extended local subperiosteal dissection. If soft tissue anchoring hooks or barbs were designed onto the implant's surface, then the overlying soft tissue is lifted and suspended onto these anchorage devices. Once everything is checked, the soft tissue margins are then closed with running sutures.

Depending upon where the implant(s) have been placed, a secondary soft tissue enhancement procedure can be performed. For example, implant placement in the pyriform rims and nasolabial areas allows for augmentation to the upper lip by performing a double V-Y closure. The patient will receive post-op instructions and a postoperative CT scan to evaluate proper placement of the implant and fixation screws. The patient will be seen one-week post-operatively for evaluation and possible suture removal. After that, the patient will be seen as [if] necessary until six months postoperative. The patient will be required to be seen at six months postoperatively, and at this time, a CT scan and color imaging will be taken (it is recommended that the scan be taken on the same machine with the same settings and field of view as the preoperative CT scan). By 6 months, most of the postoperative swelling will have resolved, and the surgeon or a technician will upload the DICOM file and the color image files to a HIPAA-compliant site. These files will be utilized and compared to the preoperative CT scan along with the prediction file, and the differences will be noted. These differences will be utilized in the refinement of the prediction algorithm.

This invention aims to provide a surgical prediction tool that will directly determine the design and manufacturing of a custom facial implant. The information collected in the preoperative CT, the patient-specific implant design process, and the six-month to 1-year postoperative CT (after the implant was placed) will refine the algorithm and make the soft tissue prediction software more accurate. The greater the number of implants designed, manufactured and implanted by this method will improve the prediction and design process going forward in an automated manner incorporating artificial intelligence.

The invention also includes utilizing developing artificial intelligence to continually enhance the soft tissue prediction software through follow-up studies. All subjects will have a CT scan and color images taken six months after the placement of the implants. This CT scan will be compared with the preoperative CT scan and the prediction plan. The specified soft tissue, bony, and implant coordinates will be entered into an algorithm that will be utilized in future facial soft tissue predictions and underlying custom implant designs.

The invention also includes custom facial implants for decreasing and arresting the natural rate of aging in site specific areas of the facial skeleton in an individual using the methodology of the invention process.

The custom facial implants are created using CADCAM and 3D printing of site specific areas of a patient's facial skeleton. The custom implants are surgically placed on the site-specific areas of the facial skeleton through a transoral approach such that there are no external incision or scars on the patients face to prevent future changes to the facial skeleton in said areas to decrease or arrest the natural aging process.

The custom facial implants are made of a non resorbable material that is safe for human implantation. A non resorbable material used in the invention is PEKK (Polyetherketoneketone) as described earlier. This material is both FDA approved and can be used in 3-D printing.

The preceding description of various and preferred embodiments of the present invention has been provided for purposes of illustration only, and it is understood that numerous modifications, variations, and alterations may be made without departing from the scope and spirit of the invention as outlined in the following claims.

What is claimed is:

1. A method of decreasing and arresting the natural rate of aging in site specific areas of the facial skeleton in an individual by utilizing custom facial implants in said site specific areas, including the steps of:
using CADCAM and 3D printing to create said custom patient facial implants;
placing said implants on said site-specific areas of the facial skeleton through a transoral approach such that there are no external incision or scars on the patients face;
surgically fixating said custom implants directly to the bone to promote integration of said implants to the underlying bone and prevent future changes to the facial skeleton in said areas to decrease or arrest the natural aging process.

2. The method according to claim 1, overlying facial soft tissue is supported and resuspended by said custom facial implant.

3. The method according to claim 1, where said site specific areas of the facial skeleton undergo resorptive changes decreasing the natural aging process.

4. The method according to claim 1, wherein said implants are made of a non-resorbable material that adds volume to said site specific area of the facial skeleton.

5. The method according to claim 1, wherein said implants integrates into the underlying bone preventing further natural aging related bone resorption.

6. The method according to claim 1, wherein said implants resuspend the soft tissue above said implant.

7. The method according to claim 1, wherein said implants add volume to facial areas where the facial bone is deficient.

8. The method according to claim 1, wherein said implants add volume to facial areas where facial fat has been lost.

9. The method according to claim 1, wherein said implants add volume to facial areas where facial fat has been redistributed and the skin has sagged.

10. The method according to claim 1, wherein the patients facial skeletal foundation is enhanced, and the individual's face has a plumper, smoother, youthful appearance.

11. The method according to claim 1, wherein said implants include hooks and/or barbs engineered onto the surface of said implants in specific areas that serve as anchorage points in supporting up lifted true periosteal dermal ligaments and the accompanying soft tissue subunits of the face.

12. Custom facial implants for decreasing and arresting the natural rate of aging in site specific areas of the facial skeleton in an individual; wherein said custom facial implants are created using CADCAM and 3D printing of site specific areas of a patients facial skeleton; wherein said custom implants are surgically placed on said site-specific areas of the facial skeleton through a transoral approach such that there are no external incision or scars on the patients face to prevent future changes to the facial skeleton in said areas to decrease or arrest the natural aging process.

13. The custom facial implants according to claim 12, wherein said facial implants are made of a non resorbable material.

14. The custom facial implants according to claim 13, wherein said non resorbable material is PEKK (Polyetherketoneketone).
